(19) United States
(12) Patent Application Publication
(10) Pub. No.: US 2024/0074811 A1
(43) Pub. Date: Mar. 7, 2024

(54) SYSTEMS AND METHODS FOR VISUALIZING ANATOMICAL STRUCTURE OF PATIENT DURING SURGERY

(71) Applicant: SHANGHAI UNITED IMAGING INTELLIGENCE CO., LTD., Shanghai (CN)

(72) Inventors: Ziyan WU, Cambridge, MA (US); Benjamin PLANCHE, Cambridge, MA (US); Meng ZHENG, Cambridge, MA (US); Arun INNANJE, Cambridge, MA (US); Terrence CHEN, Cambridge, MA (US)

(73) Assignee: SHANGHAI UNITED IMAGING INTELLIGENCE CO., LTD., Shanghai (CN)

(21) Appl. No.: 17/930,058

(22) Filed: Sep. 6, 2022

(51) Int. Cl. A61B 34/10; A61B 90/00; G06T 7/33; G06T 7/70; G06T 11/00

(52) U.S. Cl. CPC A61B 34/10; A61B 90/361; G06T 7/337; G06T 7/70; G06T 11/00; A61B 2034/105; A61B 2034/107; A61B 2090/373; G06T 2207/20212; G06T 2207/30004; G06T 2207/30201; G06T 2210/41

(57) ABSTRACT

A system and method for visualizing anatomical structure of a patient during a surgery are provided. An anatomical image and a first optical image of a patient may be obtained. The anatomical image may be captured by performing a medical scan on the patient before a surgery of the patient, and the first optical image may be captured during the surgery. A target image may be generated by combining the anatomical image and the first optical image. The target image may be rendered based on a viewpoint of a target operator of the surgery. A display device may be directed to display the rendered target image.

### TECHNICAL FIELD

The present disclosure relates to medical technology, and in particular, to systems and methods for visualizing anatomical structure of a patient during a surgery.

### BACKGROUND

During a surgery of a patient, an anatomical image showing the anatomical structure of the patient often needs to be displayed for providing guidance to a user (e.g., a surgeon). The display mode of the anatomical image may affect the effect and the efficiency of the surgery.

### SUMMARY

According to an aspect of the present disclosure, a system for visualizing anatomical structure of a patient during a surgery may be provided. The system may include at least one storage device including a set of instructions and at least one processor. The at least one processor may be configured to communicate with the at least one storage device. When executing the set of instructions, the at least one processor may be configured to direct the system to perform one or more of the following operations. The system may obtain an anatomical image and a first optical image of a patient. The anatomical image may be captured by performing a medical scan on the patient before a surgery of the patient, and the first optical image may be captured during the surgery. The system may generate a target image by combining the anatomical image and the first optical image. The system may also render the target image based on a viewpoint of a target operator of the surgery. The system may further direct a display device to display the rendered target image.

In some embodiments, to generate a target image by combining the anatomical image and the first optical image, the system may obtain a reference optical image of the patient captured during the medical scan. The system may also transform the anatomical image based on the reference optical image and the first optical image such that a posture of the patient in the transformed anatomical image aligns with the posture of the patient in the first optical image. The system may generate the target image by combining the transformed anatomical image and the first optical image.

In some embodiments, to transform the anatomical image based on the reference optical image and the first optical image, the system may determine a first transformation relationship between the reference optical image and the anatomical image. The system may also determine a second transformation relationship between the reference optical image and the first optical image. The system may further transform the anatomical image based on the first transformation relationship and the second transformation relationship such that the posture of the patient in the transformed anatomical image aligns with the posture of the patient in the first optical image.

In some embodiments, to determine a second transformation relationship between the reference optical image and the first optical image, the system may determine a reference representation relating to the posture of the patient in the reference optical image based on the reference optical image. The system may also determine a target representation relating to the posture of the patient in the first optical image based on the first optical image. The system may further determine the second transformation relationship based on the reference representation and the target representation of the patient.

In some embodiments, the reference representation and the target representation of the patient may be obtained using a representation determination model.

In some embodiments, to generate the target image by combining the transformed anatomical image and the first optical image, the system may determine a field of view (fov) of the target operator based on the viewpoint of the target operator. The system may also determine a target portion of the transformed anatomical image corresponding to at least a portion of the patient within the fov of the target operation. The system may further generate the target image by overlaying the target portion of the transformed anatomical image on the first optical image.

In some embodiments, to determine the viewpoint of the target operator, the system may determine facial information of the target operator based on a second optical image of the target operator. The system may further determine the viewpoint of the target operator based on the facial information.

In some embodiments, to render the target image based on a viewpoint of a target operator of the surgery, the system may obtain a third optical image of a surgical device used in the surgery captured when the first optical image is captured. The system may also process the target image by adding a visual element representing the surgical device on the target image based on the third optical image of the surgical device. The system may further render the processed target image based on the viewpoint of the target operator.

In some embodiments, to render the target image based on a viewpoint of a target operator of the surgery, the system may obtain a fourth optical image of one or more markers placed on the patient captured when the first optical image is captured. The system may also process the target image by adding a visual element representing the one or more markers on the target image based on the fourth optical image of the one or more markers. The system may further render the processed target image based on the viewpoint of the target operator.

In some embodiments, the viewpoint of the target operator may be set by the target operator.

According to another aspect of the present disclosure, a method for visualizing anatomical structure of a patient during a surgery may be provided. The method may include obtaining an anatomical image and a first optical image of a patient. The anatomical image may be captured by performing a medical scan on the patient before a surgery of the patient, and the first optical image may be captured during the surgery. The method may include generating a target image by combining the anatomical image and the first optical image. The method may also include rendering the target image based on a viewpoint of a target operator of the surgery. The method may further include directing a display device to display the rendered target image.

According to another aspect of the present disclosure, a non-transitory computer readable medium. The non-transitory computer readable medium may include at least one set of instructions for visualizing anatomical structure of a patient during a surgery. When executed by one or more processors of a computing device, the at least one set of instructions may cause the computing device to perform a method. The method may include obtaining an anatomical image and a first optical image of a patient. The anatomical image may be captured by performing a medical scan on the patient before a surgery of the patient, and the first optical image may be captured during the surgery. The method may include generating a target image by combining the anatomical image and the first optical image. The method may also include rendering the target image based on a viewpoint of a target operator of the surgery. The method may further include directing a display device to display the rendered target image.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an exemplary application scenario of a surgery system according to some embodiments of the present disclosure;

FIG. 2 is a block diagram illustrating an exemplary surgery system according to some embodiments of the present disclosure;

FIG. 3A is a block diagram illustrating exemplary processing device according to some embodiments of the present disclosure;

FIG. 3B is a block diagram illustrating another exemplary processing device according to some embodiments of the present disclosure;

FIG. 4 is a flowchart illustrating an exemplary process for visualizing an anatomical structure of a patient during a surgery of the patient according to some embodiments of the present disclosure;

FIG. 5 is a flowchart illustrating an exemplary process for transforming an anatomical image of a patient according to some embodiments of the present disclosure; and

FIG. 6 is a flowchart illustrating an exemplary process for generating a representation determination model according to some embodiments of the present disclosure.

### DETAILED DESCRIPTION

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

In the following detailed description, numerous specific details are set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, in order to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is for the purpose of describing particular example embodiments only and is not intended to be limiting. As used herein, the singular forms “a,” “an,” and “the” may be intended to include the plural forms as well, unless the context clearly indicates otherwise. It will be further understood that the terms “comprise,” “comprises,” and/or “comprising,” “include,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It will be understood that the term “system,” “engine,” “unit,” “module,” and/or “block” used herein are one method to distinguish different components, elements, parts, sections or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or from themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption prior to execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks, but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that when a unit, engine, module, or block is referred to as being “on,” “connected to,” or “coupled to,” another unit, engine, module, or block, it may be directly on, connected or coupled to, or communicate with the other unit, engine, module, or block, or an intervening unit, engine, module, or block may be present, unless the context clearly indicates otherwise. As used herein, the term “and/or” includes any and all combinations of one or more of the associated listed items. The term “pixel” and “voxel” in the present disclosure are used interchangeably to refer to an element of an image. An anatomical structure shown in an image of a subject (e.g., a patient) may correspond to an actual anatomical structure existing in or on the subject's body.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings are for the purpose of illustration and description only and are not intended to limit the scope of the present disclosure. It is understood that the drawings are not to scale.

Conventionally, medical scan(s) are performed on a patient before and/or during a surgery of the patient to visualize the anatomical structure of the patient to provide guidance for an operator (e.g., a surgeon) (or referred to as a user) who performs the surgery. In order to obtain a relatively good visualization result, conventional approaches usually require the patient to keep the same pose during the surgery as he/she was during the medical scan(s), which is difficulty. By aligning markers on a surface of the patient manually or automatically, anatomical image(s) captured by the medical scan(s) may be registered to an actual body surface of the patient in a same coordinate system, and the registered anatomical image(s) can be visualized via an augmented reality headset. However, wearing an augmented reality headset during the surgery may be cumbersome and the jittering effect caused by localization inaccuracy could impact the user experience and the overall efficacy of the surgery guidance. In addition, the registration between the anatomical image(s) and the body surface of the patient based on the markers is often coarse and inaccurate due to a difference between the patient posture in the surgery and the patient posture in the medical scan(s). Some other conventional approaches may perform a medical scan during the surgery to visualize the anatomical structure of the patient, which results in additional radiation dosage to the patient.

An aspect of the present disclosure relates to systems and methods for visualizing an anatomical structure of a patient during a surgery of the patient. The systems may obtain an anatomical image and a first optical image of the patient. The anatomical image may be captured by performing a medical scan on the patient before the surgery of the patient and the first optical image may be captured during the surgery. The systems may generate a target image by combining the anatomical image and the first optical image.

In some embodiments, the anatomical image may be transformed based on a reference optical image and the first optical image such that a posture of the patient in the transformed anatomical image aligns the posture of the patient in the first optical image, and the target image may be generated by combining the transformed anatomical image and the first optical image. The systems may also render the target image based on the viewpoint of the target operator of the surgery when the first optical image is captured. The systems may further direct a display device to display the rendered target image to the target operator.

Some embodiments of the present disclosure may reduce or eliminate the effect of the posture difference between the patient posture in the anatomical image and the patient posture in the surgery based on the first optical image and the reference optical image of the patient. In this way, the quality of the target image, which is the combination of the first optical image and the anatomical image, may be improved. The target image may illustrate both the internal structure and the body surface of the patient, and can provide better guidance for the surgery than pure anatomical images or pure optical images. In addition, the target image may be rendered according to the viewpoint of the target operator, and the rendered target image may reflect the real surgical scene seen by the target operator, therefore, the target operator may quickly perform an appropriate next surgical operation according to the rendered target image.

Compared with conventional approaches that involve multiple medical scans before and during the surgery, the methods of the present disclosure may reduce a count of the medical scans, thereby reducing adverse effects (e.g., radiation dosage) on the patient, and improving the efficiency of the generation of the target image. Moreover, the methods of the present disclosure do not require the patient to keep the same pose in the surgery as he/she was during the pre-operative medical scan. Compared with conventional approaches that align the anatomical image of the patient to the body surface of the patient in the surgery based on markers on the surface of the patient, the methods of the present disclosure based on optical image(s) may have an improved accuracy because the optical image(s), such as depth images and/or point cloud image(s), can provide more accurate information for reducing or eliminating the effect of the posture difference.

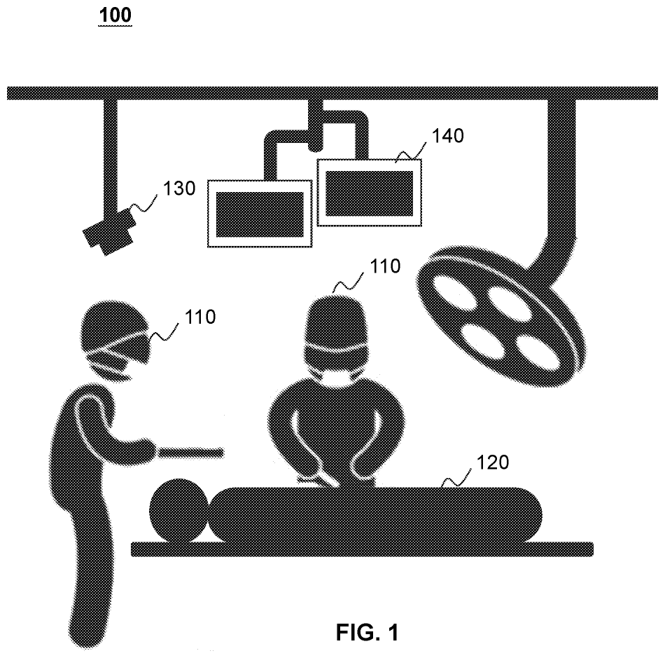

FIG. 1 is a schematic diagram illustrating an exemplary application scenario 100 of a surgery system according to some embodiments of the present disclosure.

As shown in FIG. 1, in the application scenario 100, one or more operators 110 (e.g., a surgeon) may participate in a surgery on a patient 120, and the surgery system may be configured to provide guidance to the operator(s) 110 during the surgery. For example, the surgery system may provide one or more images that visualizes an anatomical structure of the patient 120 to the operator(s).

In some embodiments, the surgery system may obtain an anatomical image of the patient 120 that illustrates an internal structure of the patient (or a portion thereof). The anatomical image may be captured by performing a medical scan on the patient 120 before or during the surgery of the patient 120. The surgery system may also obtain a first optical image of the patient 120 that illustrates an external body surface of the patient 120 (or a portion thereof). The first optical image may be captured during the surgery of the patient 120 using a visual sensor 130 mounted in a surgical room where the surgery is performed. The anatomical image and the first optical image may both include a portion of the patient that needs to receive the surgery. The surgery system may combine the anatomical image and the first optical image to generate a target image. In some embodiments, the surgery system may overlay a target portion of the anatomical image on the first optical image to generate a target image. For example, the surgery system may overlay the portion of the patient that needs to receive the surgery in the anatomical image on a corresponding portion of the first optical image to generate the target image. The generated target image may illustrate the internal structure of the portion of the patient that needs to receive the surgery, and the body surface of the other portion of the patient.

In some embodiments, the target image may be displayed on a display device 140. Optionally, the surgery system may render the target image based on a viewpoint of a target operator of the operator(s) 110 of the surgery when the first optical image is captured. For example, the rendered target image may display both the internal structure and the body surface of the patient from the perspective of the target operator. Based on the rendered target image, the target operator may have an experience of seeing through the body surface of the patient and viewing the internal structure of the patient from his/her perspective. The surgery system may further display the rendered target image to the target operator via the display device 140 to provide guidance to the target operator.

In some embodiments, the surgery may be a robotic surgery performed by a surgical robot under the supervision of the operator(s), an artificial surgery performed by the operator(s), or a mixed surgery performed by the surgical robot and the operator(s) together. In some embodiments, the surgery may include any surgery performed on the internal structure of the patient 120, such as a surgery for removing nodules in the lungs of the patient, an endoscopy surgery of the thoracic cavity of the patient, etc. In some embodiments, the operator(s) 110 may include a surgeon who performs the surgery, an assistant surgeon, an anesthesiologist, etc.

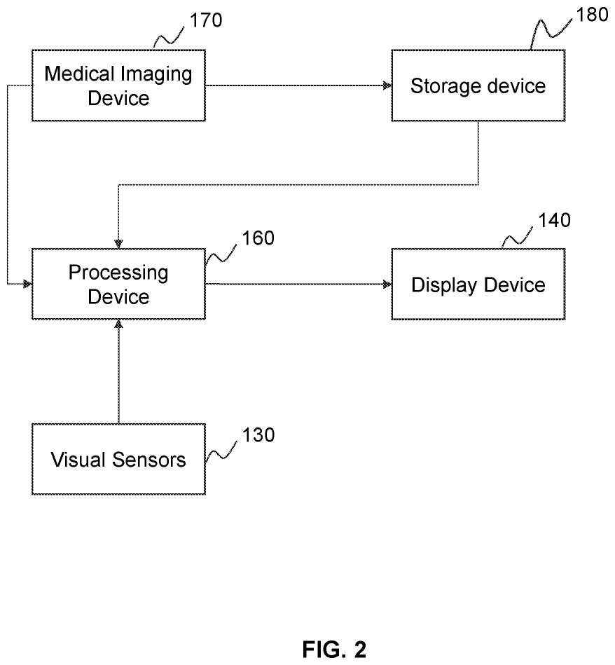

FIG. 2 is a block diagram illustrating an exemplary surgery system 200 according to some embodiments of the present disclosure. As shown in FIG. 2, the surgery system 200 may include one or more visual sensors 130, a display device 140, a processing device 160, a medical imaging device 170, and a storage device 180, etc. In some embodiments, the one or more visual sensors 130, the display device 140, the processing device 160, the medical imaging device 170, and/or storage device 180 may be connected to and/or communicate with each other via a wireless connection, a wired connection, or a combination thereof. The connections between the components in the surgery system 200 may be variable. For example, the medical imaging device 170 may be connected to the processing device 160 through a network. As another example, the medical imaging device 170 may be connected to the processing device 160 directly.

The medical imaging device 170 may be configured to scan a patient (or a part of the patient) to acquire medical image data associated with the patient before or during a surgery of the patient. The medial image data relating to the patient may be used for generating an anatomical image of the patient. The anatomical image may illustrate an internal structure of the patient. In some embodiments, the medical imaging device 170 may include a single-modality scanner and/or multi-modality scanner). The single modality scanner may include, for example, an X-ray scanner, a CT scanner, a magnetic resonance imaging (MRI) scanner, an ultrasonography scanner, a positron emission tomography (PET) scanner, a Digital Radiography (DR) scanner, or the like, or any combination thereof. The multi-modality scanner may include, for example, an X-ray imaging-magnetic resonance imaging (X-ray-MRI) scanner, a positron emission tomography-X-ray imaging (PET-X-ray) scanner, a single-photon emission computed tomography-magnetic resonance imaging (SPECT-MRI) scanner, a positron emission tomography-computed tomography (PET-CT) scanner, etc. It should be noted that the medical imaging device 170 described below is merely provided for illustration purposes, and not intended to limit the scope of the present disclosure.

The visual sensor(s) 130 may be configured to capture an optical image of the patient, which may illustrate an external body surface of the patient. For example, the visual sensor(s) 130 may be configured to capture optical images of the patient before, during, and/or after the surgery of the patient. As another example, the visual sensor(s) 130 may be configured to capture optical images of the patient during the scan of the patient performed by the medical imaging device 170. The visual sensor(s) 130 may be and/or include any suitable device capable of capturing optical images of subjects located in a field of view of the visual sensor(s) 130. For example, the visual sensor(s) 130 may include a camera (e.g., a digital camera, an analog camera, a binocular camera, etc.), a red-green-blue (RGB) sensor, an RGB-depth (RGB-D) sensor, a time-of-flight (TOF) camera, a depth camera, a structure light camera, or the like, or any combination thereof. In some embodiments, the optical image(s) captured by the visual sensor(s) may include three-dimensional surface information of the patient, such as depth information, point cloud information, TOF information, or the like, or any combination thereof.

The display device 140 may be configured to display information received from other components of the surgery system 200. For example, the display device 140 may display image(s) received from the processing device 160 to a target operator (e.g., a surgeon who performs the surgery of the patient). The display device 140 may include a liquid crystal display (LCD), a light emitting diode (LED)-based display, a flat panel display or curved screen (or television), a cathode ray tube (CRT), or the like, or a combination thereof. In some embodiments, the display device 140 may be an immersive display, such as, a virtual reality device, an augmented reality device, a mixed reality device, etc., worn by a subject (e.g., the target operator 110). For example, the immersive display may be a head-mounted display. The head-mounted display may be a set of glasses or goggles covering the user's eyes. As another example, the display device 140 may be an augmented reality device that overlays the target image on the real world (e.g., the real patient).

The display device 140 may be mounted in any suitable location in a surgery room for the surgery of the patient. For example, the display device may be located in a position opposite the target operator, so that the target operator views the rendered target image easily. In some embodiments, the display device may be movable. For example, the position of the display device may be adjusted automatically by the processing device 160A according to a position of the target operator. Alternatively, the position of the display device may be adjusted manually by a user (e.g., the target operator or another operator participating in the surgery). In this way, when the position of the target operator is changed, the position of the display device can also be changed accordingly, so that the target operator may browse the rendered target image easily. In some embodiments, multiple display devices may be installed at different locations in the surgery room, so that the target operator can choose one display device to browse the rendered target image according to his/her location and/or preferences.

The storage device 180 may be configured to store data, instructions, and/or any other information. The storage device 180 may store data obtained from the visual sensor(s) 130, the display device 140, the processing device 160, and the medical imaging device 170. For example, the storage device 180 may store optical images captured by the visual sensor(s) 130 and/or anatomical images captured by the medical imaging device 170. In some embodiments, the storage device 180 may store data and/or instructions that the processing device 160 may execute or use to perform exemplary methods described in the present disclosure.

The processing device 160 may be configured to process data and/or information obtained from one or more components (e.g., the visual sensor(s) 130, the display device 140, the medical imaging device 170, the storage device 180) of the surgery system 200. For example, the processing device 160 may obtain an anatomical image from the medical imaging device 170 or the storage device 180, and obtain a first optical image of the patient from the visual sensor(s) 130 or the storage device 180. The anatomical image may be captured by performing a medical scan on the patient before the surgery of the patient, and the first optical image may be captured during the surgery of the patient. The processing device 160 may generate a target image by combining the anatomical image and the first optical image. In some embodiments, the target image may be transmitted to the display device 140 for display. Optionally, the processing device 160 may render the target image based on a viewpoint of a target operator of the surgery when the first optical image is captured so that the target image displays the internal structure and/or the body surface of the patient from the viewpoint of the target operator. In some embodiments, the target image may be updated in the surgery when, for example, the patient moves and/or the viewpoint of the target operator changes. In some embodiments, the processing device 160 may be a single server or a server group. The server group may be centralized or distributed. In some embodiments, the processing device 160 may be local or remote.

This description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. However, those variations and modifications do not depart the scope of the present disclosure. Merely by way of example, the surgery system 200 may include one or more additional components and/or one or more components described above may be omitted. For example, the surgery system 200 may include a network. The network may include any suitable network that can facilitate the exchange of information and/or data for the surgery system 200. In some embodiments, one or more components of the surgery system 200 (e.g., the visual sensor(s) 130, the medical imaging device 170, the processing device 160, the display 140, etc.) may communicate information and/or data with one or more other components of the surgery system 200 via the network. It also should be understood that the connections between the components FIG. 2 are illustrative and can be modified. For example, the visual sensor(s) 130 may be connected to the storage device 180.

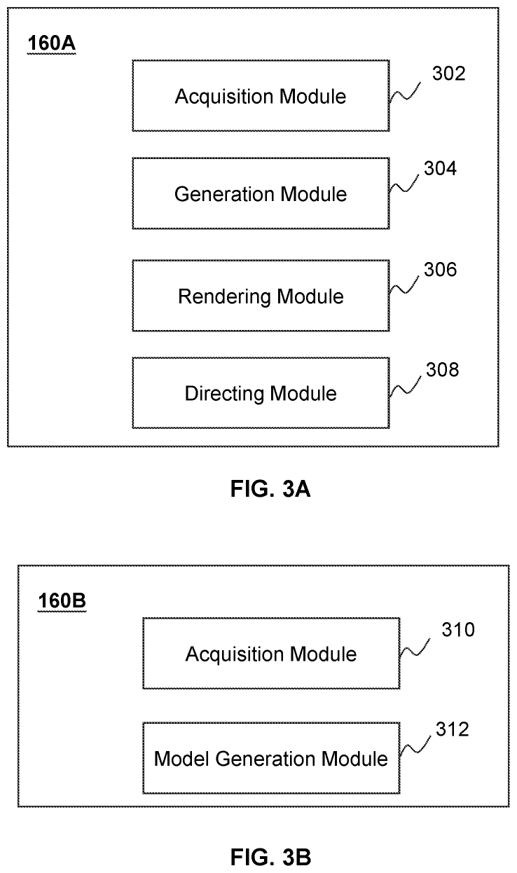

FIG. 3A is a block diagram illustrating exemplary processing device 160A according to some embodiments of the present disclosure. FIG. 3B is a block diagram illustrating another exemplary processing device 160B according to some embodiments of the present disclosure. The processing devices 160A and 160B may be exemplary processing devices 160 as described in connection with FIG. 2. In some embodiments, the processing device 160A may be configured to perform methods for surgery guidance disclosed herein. The processing device 160B may be configured to generate one or more machine learning models that can be used in the surgery guidance methods. In some embodiments, the processing devices 160A and 160B may be respectively implemented on a computing device. Alternatively, the processing devices 160A and 160B may be implemented on a same computing device.

As shown in FIG. 3A, the processing device 160A may include an acquisition module 302, a generation module 304, a rendering module 306, and a directing module 308.

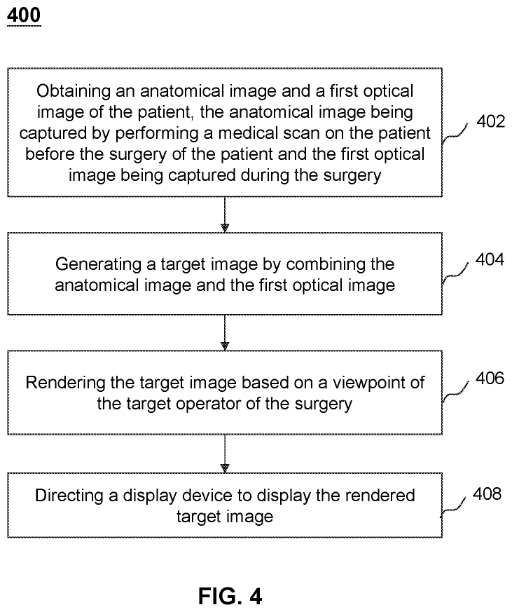

The acquisition module 302 may be configured to obtain information relating to the surgery system 200. For example, the acquisition module 302 may obtain an anatomical image and a first optical image of the patient. the anatomical image may be captured by performing a medical scan on the patient before the surgery of the patient and the first optical image may be captured during the surgery. More descriptions regarding the obtaining of the anatomical image and the first optical image of the patient may be found elsewhere in the present disclosure. See, e.g., operation 402 in FIG. 4, and relevant descriptions thereof.

The generation module 304 may be configured to generate a target image by combining the anatomical image and the first optical image. The generated target image may be regarded as a combination of the anatomical image and the first optical image, and provide information regarding both the internal structure and the body surface of the patient. The target image may illustrate relative positions between internal organs inside the patient, and relative positions between the internal organs and the body surface, thereby facilitating the target operation to perform the surgery. More descriptions regarding the generation of the target image may be found elsewhere in the present disclosure. See, e.g., operation 404 in FIG. 4, and relevant descriptions thereof.

The rendering module 306 may be configured to render the target image based on a viewpoint of the target operator of the surgery. The target operator may be any operator who participates in the surgery, for example, an operator who performs or assists the surgery of the patient. More descriptions regarding the rendering of the target image based on the viewpoint of the target operator of the surgery may be found elsewhere in the present disclosure. See, e.g., operation 406 in FIG. 4, and relevant descriptions thereof.

The directing module 308 may be configured to direct a display device to display the rendered target image. More descriptions regarding the directing of the display device to display the rendered target image may be found elsewhere in the present disclosure. See, e.g., operation 408 in FIG. 4, and relevant descriptions thereof.

As shown in FIG. 3B, the processing device 160B may include an acquisition module 310 and a model generation module 312.

The acquisition module 310 may be configured to obtain data used to train a machine learning model, such as a representation determination model. For example, the acquisition module 310 may be configured to obtain one or more training samples. More descriptions regarding the acquisition of the training samples may be found elsewhere in the present disclosure. See, e.g., operations 602 in FIG. 6, and relevant descriptions thereof.

The model generation module 312 may be configured to generate the representation determination model by model training. In some embodiments, the representation determination model may be generated according to a machine learning algorithm. More descriptions regarding the generation of the representation determination model may be found elsewhere in the present disclosure. See, e.g., operation 604 in FIG. 6, and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, the processing device 160A as described in FIG. 3A and/or the processing device 160B as described in FIG. 3B may share two or more of the modules, and any one of the modules may be divided into two or more units. For instance, the processing device 160A as described in FIG. 3A and the processing device 160B as described in FIG. 3B may share a same acquisition module; that is, the acquisition module 302 and the acquisition module 310 are a same module. In some embodiments, the processing device 160A as described in FIG. 3A and/or the processing device 160B as described in FIG. 3B may include one or more additional modules, such as a storage module (not shown) for storing data. In some embodiments, the processing device 160A as described in FIG. 3A and the processing device 160B as described in FIG. 3B may be integrated into one processing device 160.

FIG. 4 is a flowchart illustrating an exemplary process 400 for visualizing an anatomical structure of a patient during a surgery of the patient according to some embodiments of the present disclosure. In some embodiments, the process 400 may be implemented in the surgery system 200 illustrated in FIG. 2. For example, the process 400 may be stored in the storage device 180 of the surgery system 200 as a form of instructions, and invoked and/or executed by the processing device 160A (e.g., one or more modules as illustrated in FIG. 3A). The operations of the illustrated process presented below are intended to be illustrative. In some embodiments, the process 400 may be accomplished with one or more additional operations not described, and/or without one or more of the operations discussed. Additionally, the order in which the operations of the process 400 as illustrated in FIG. 4 and described below is not intended to be limiting.

In 402, the processing device 160A (e.g., the acquisition module 302) may obtain an anatomical image and a first optical image of the patient, the anatomical image being captured by performing a medical scan on the patient before the surgery of the patient and the first optical image being captured during the surgery.

As used herein, an anatomical image of the patient refers to an image illustrating an internal structure of the patient (or a portion thereof), and an optical image of the patient refers to an image illustrating an external body surface of the patient (or a portion thereof). In some embodiments, the anatomical image may illustrate the internal structure of a target region of the patient and optionally surrounding regions of the target region. Additionally or alternatively, the first optical image may illustrate the body surface of the target region and optionally surrounding regions of the target region.

As used herein, the target region of the patient refers to a region of the patient that needs to receive the surgery. For example, the target region may include the lungs of the patient including nodules that need to be removed. As another example, the target region may include the stomach of the patient that needs to receive a gastric endoscopy.

In some embodiments, the anatomical image may include a 2D image (e.g., a slice image), a 3D image, or the like. In some embodiments, the anatomical image may include a medical image generated by a biomedical imaging technique as described elsewhere in this disclosure. For example, the anatomical image may include an MRI image, a PET image, a CT image, a PET-CT image, a PET-MR image, an ultrasound image, etc.

In some embodiments, the anatomical image may be generated based on image data acquired using the medical imaging device 170 of the surgery system 200 or an external imaging device. For example, the medical imaging device 170, such as a CT device, an MRI device, an X-ray device, a PET device, or the like, may be directed to scan the patient or a portion of the patient (e.g., the chest of the patient). The processing device 160A may generate the anatomical image based on the image data acquired by the medical imaging device 170. In some embodiments, the anatomical image may be previously generated and stored in a storage device (e.g., a storage device of the surgery system 200, or an external source). The processing device 160A may retrieve the anatomical image from the storage device.

In some embodiments, the first optical image may be captured by a first visual sensor (e.g., the visual sensor(s) 130). The first visual sensor may be mounted on the surgical room where the surgery performs and directed to the patient (e.g., the target region of the patient). For example, the first optical image may be a real-time first optical image captured by the first visual sensor. As used herein, the term “real-time” refers to that a time interval between when an optical image is captured by the first visual sensor and when the optical image is obtained by the processing device 160A for analysis is smaller than a time threshold.

In some embodiments, the first visual sensor may capture optical images of the patient during the surgery continuously or intermittently (e.g., periodically). Each time the first visual sensor captures an optical image, the first visual sensor may transmit the optical image to the processing device 160A as the first optical image for analysis.

In some embodiments, the anatomical image may be captured by performing a medical scan on the patient during the surgery of the patient. The first optical image may be captured later than the anatomical image.

In some embodiments, the anatomical image and the first optical image may be 3D images. Alternatively, the anatomical image and the first optical image may be 2D images. In some embodiments, the anatomical image and the first optical image may be 3D images, and the processing device 160A may project the 3D anatomical image and the 3D first optical image to a specified 2D viewing plane to obtain a 2D anatomical image and a 2D first optical image. The 2D viewing plane may be determined by the surgery system 200 or a user (e.g., the target operator).

In 404, the processing device 160A (e.g., the generation module 304) may generate a target image by combining the anatomical image and the first optical image.

In some embodiments, the processing device 160A may generate the target image by overlaying a target portion (also referred to as a first target portion) of the anatomical image on the first optical image. The target portion of the anatomical image may include the whole anatomical image or a portion of the anatomical image. For example, the target portion may correspond to the target region of the patient. As another example, the target portion of the anatomical image may correspond to the target region of the patient and optionally surrounding regions of the target region. As still another example, the target portion of the anatomical image may correspond to the whole patient. In some embodiments, the processing device 160A may generate the target image by overlaying a second target portion of the first optical image on the anatomical image. The second target portion of the first optical image may include the whole first optical image or a portion of the first optical image. For example, the second target portion may correspond to the target region of the patient and optionally surrounding regions of the target region. As another example, the second target portion of the first optical image may correspond to the whole patient. The generated target image may be regarded as a combination of the anatomical image and the first optical image, and provide information regarding both the internal structure and the body surface of the patient. The target image may illustrate relative positions between internal organs inside the patient, and relative positions between the internal organs and the body surface, thereby facilitating the target operation to perform the surgery. For example, the target operator may locate the target region of the patient (i.e., the surgery site) based on the target image. For illustration purposes, the generation of the target image by overlaying the target portion of the anatomical image on the first optical image is described as an example.

In some embodiments, the processing device 160A may identify the target portion of the anatomical image and a portion corresponding to the target portion in the first optical image. The processing device 160A may overlay the target portion of the anatomical image on the portion corresponding to the target portion in the first optical image to generate the target image. However, since a posture of the patient in the anatomical image may be different from the posture of the patient in the first optical image, the target image may have a poor quality (e.g., a mismatch between the internal structure and the body surface if the target portion of the anatomical image is directly overlaid on the first optical image).

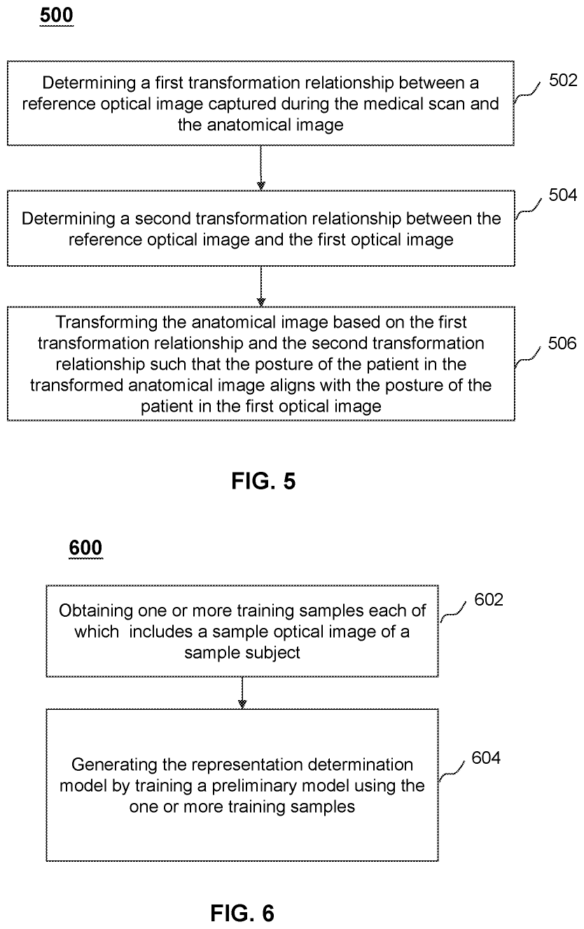

In order to reduce or eliminate the effect of the posture difference, the processing device 160A may transform the anatomical image such that the posture of the patient in the transformed anatomical image is consistent with the posture of the patient in the first optical image. In some embodiments, the processing device 160A may obtain a reference optical image of the patient captured during the medical scan, and transform the anatomical image based on the reference optical image. More descriptions for the transformation of the anatomical image may be found elsewhere in the present disclosure (e.g., FIG. 5 and the descriptions thereof).

The processing device 160A may further generate the target image by combining the transformed anatomical image and the first optical image. For example, a target portion of the transformed anatomical image may be determined by the processing device 160A, and overlaid on the first optical image to generate the target image. For example, the target portion of the transformed anatomical image may correspond to the target region of the patient. As another example, the target portion of the transformed anatomical image may correspond to a portion of the target region of the patient that is within the FOV of the target operation.

The processing device 160A may generate the target image by overlaying the target portion of the transformed anatomical image on the first optical image. For example, the processing device 160A may determine a specific portion in the first optical image corresponding to the target portion of the transformed anatomical image. The specific portion identified in the first optical image may correspond to the same body part as the target portion of the transformed anatomical image, for example, the specific portion of the first optical image and the target portion of the transformed anatomical image both correspond to the target region of the patient. Then, the processing device 160A may overlay the target portion of the transformed anatomical image on the specific portion of the first optical image to generate the target image.

In 406, the processing device 160A (e.g., the rendering module 306) may render the target image based on a viewpoint of the target operator of the surgery.

The target operator may be any operator who participates in the surgery, for example, an operator who performs or assists the surgery of the patient. In some embodiments, the processing device 160A may automatically determine the target operator by analyzing activities and behaviors of each person in the surgical room where the surgery of the patient performs based on the first optical image. For example, the processing device 160A may identify, from the first optical image, a person who is performing the surgery of the patient as the target operator. Alternatively or optionally, the target operator may be determined by a user manually.

The viewpoint of the target operator refers to a visual angle of the eyes of the target operator at which content is viewed. For example, the viewpoint of the target operator may be represented by a gaze direction, a head orientation, or the like, or any combination thereof.

In some embodiments, the processing device 160A may determine the viewpoint of the target operator by processing a second optical image of the target operator using one or more image recognition algorithms. The first optical image and the second optical image may be the same optical image if the first optical image includes both the patient and the target operator. That is, the viewpoint of the target operator may be determined based on the first optical image. Alternatively, the second optical image may be a different image from the first optical image. For example, the second optical image may be captured by a second visual sensor directed to the target operator, and the shooting times of the first optical image and the second optical image may be the same. The second visual sensor and the first visual sensor may be of the same type or different types.

In some embodiments, the second optical image may be an optical image in which the target operator is looking at the patient. For example, during the surgery, the target operator may look at other objects in the operation room, for example, the target operator may browse target images on a display, communicate with other operators (e.g., an assistant), etc. The viewpoint of the target operator may need to be determined based on an optical image in which the target operator is looking at the patient.

In some embodiments, optical images of the target operator may be captured continuously or intermittently (e.g., periodically) during the surgery, and transmitted to the processing device 160A. Each time the processing device 160A receives an optical image of the target operator, the processing device 160A may determine whether the received optical image is a second optical image. If the received optical image of the target operator is a second optical image, the processing device 160A may determine or update the viewpoint of the target operator based on the received optical image. If the received optical image of the target operator is not a second optical image, the processing device 160A may not update the viewpoint, that is, the viewpoint of the target operator may be determined based on a previous second optical image.

For example, the processing device 160A may determine whether the FOV of the target operator covers at least a portion of the patient (e.g., the target region of the patient) in the received optical image. In response to determining that the FOV of the target operator covers at least a portion of the target region of the patient, the processing device 160A may determine that the received optical image is a second optical image. In response to determining that the FOV of the target operator does not cover at least a portion of the patient, the processing device 160A may determine that the received optical image is not a second optical image.

As another example, the processing device 160A determine whether the received optical image is a second optical image based on activities of hands of the target operator in the received optical image. Merely by way of example, based on the received optical image, the processing device 160A may determine whether the target operator is performing a surgical procedure on the patient when the received optical image is captured. In response to determining that the target operator is performing a surgical procedure on the patient, the processing device 160A determine that the received optical image is a second optical image. In response to determining that the target operator is not performing a surgical procedure on the patient, the processing device 160A determine that the received optical image is not a second optical image.

In some embodiments, whether the received optical image is a second optical image may be determined in any suitable manner, for example, based on the head orientation of the target operator when the received optical image is captured.

In some embodiments, the processing device 160A may determine facial information of the target operator based on the second optical image. Exemplary facial information of the target operator may include locations of the eyes, gaze directions of the eyes, a head orientation, a normal direction of the face, or the like, or any combination thereof, of the target operator. The processing device 160A may further determine the viewpoint of the target operator based on the facial information. For example, the processing device 160A may determine the viewpoint of the target operator according to the locations and the gaze directions of the eyes of the target operator.

In some embodiments, the viewpoint of the target operator may be determined based on the second optical image via detecting one or more apparatuses (e.g., glasses, googles, etc.) that the target operator is wearing. For example, the processing device 160A may determine information relating to the glasses that the target operator is wearing based on the second optical image. Exemplary information relating to the glasses may include a location of the glasses, a normal direction of the lenses of the glasses, or the like, or any combination thereof. The processing device 160A may determine the viewpoint of the target operator based on the information relating to glasses.

In some embodiments, the second visual sensor for capturing the second optical image may be mounted on a display device for displaying the rendered target image described in operation 408. Alternatively, the second visual sensor may be mounted on other objects in the surgery room, such as a wall, the ceiling, or a fixture.

In some embodiments, the viewpoint of the target operator may be set or adjusted by a user (e.g., the target operator himself/herself) manually. For example, the target operator may directly set the viewpoint of the target operator through an input device such as a keyboard, a joystick, etc. As another example, if the target operator considers that the viewpoint determined by the processing device 160A has low accuracy, the target operator may modify the viewpoint determined by the processing device 160A according to actual needs.

Further, the processing device 160A may render the target image based on the viewpoint of the target operator.

In some embodiments, the processing device 160A may render the target image by rotating the target image based on the viewpoint of the target image. The rendered target image may display the internal structure and the body surface of the patient from the perspective of the target operator.

In some embodiments, the processing device 160A may process the target image, and then rotate the processed target image based on the viewpoint of the target operator, wherein the rotated processed target image may be designated as the rendered target image. The processing of the target image may include, for example, a geometric transformation, a projection transformation, a perspective transformation, a window clipping, assigning materials, coloring, or the like, or any combination thereof.

For example, the processing device 160A may segment, from the target image, one or more subregions each of which corresponds to an organ or tissue of the patient that is located in the target region according to an image analysis algorithm (e.g., an image segmentation algorithm). For example, the processing device 160A may perform image segmentation on the target image using an image segmentation algorithm. Exemplary image segmentation algorithms may include a thresholding segmentation algorithm, a compression-based algorithm, an edge detection algorithm, a machine learning-based segmentation algorithm, or the like, or any combination thereof. In some embodiments, each of the subregion(s) may be segmented from the target image manually by a user (e.g., the target operator, an imaging specialist, a technician) by, for example, drawing a bounding box on the subregion displayed on a user interface. The one or more subregions and the remaining region of the target image may be assigned with different colors to differentiate different regions of the patient in the resulting rendered target image so that the target operator may quickly and accurately find an organ or tissue based on the rendered target image. For example, the processing device 160A may color each subregion in the target image using the determined color corresponding to the subregion, wherein the colored target image may be deemed as the processed target image.

In some embodiments, the processing device 160A may obtain a third optical image of a surgical device used in the surgery captured by a third visual sensor when the first optical image is captured. The third optical image may include information regarding, for example, the appearance of the surgical device, the position of the surgical device relative to the patient, the orientation of the surgical device, or the like, or any combination thereof. The processing device 160A may process the target image by adding a visual element representing the surgical device on the target image based on the third optical image of the surgical device. The visual element representing the surgical device may be, for example, an icon representing the surgical device, a region in a shape of the surgical device, a real image of the surgical device, etc. In some embodiments, the third optical image of the surgical device and the first optical image may be the same optical image. The processing device 160A may directly generate the visual element of the surgical device according to the first optical image.

In some embodiments, the third optical image of the surgical device and the first optical image may be different optical images. The processing device 160A may obtain a transformation relationship between a first coordinate system corresponding to the third optical image (e.g., a camera coordinate system of the third visual sensor) and a second coordinate system corresponding to the first optical image (e.g., a camera coordinate system of the first visual sensor). The processing device 160A may determine a region in the target image corresponding to the surgical device according to the transformation relationship between the first coordinate system and the second coordinate system. The processing device 160A may add the visual element representing the surgical device on the region in the target image corresponding to the surgical device.

In some embodiments, since the rendered target image illustrates the internal structure of the patient, the rendered target image including the visual element of the surgical device may provide reference information for the target operator to determine a suitable location of the surgical device. For example, the target operator may try to move the surgical device to different positions relative to the patient, and real-time third optical images of the surgical device may be captured and transmitted to the processing device 160A. Based on the latest obtained third optical image, the processing device 160A may update the position and/or the orientation of the visual element representing the surgical device in the processed target image. The processing device 160A may further render the processed target image based on the viewpoint of the target operator. In this way, the rendered target image may display the relative position of the surgical device to the internal structure of the patient (e.g., an internal target organ to be removed) in real-time, so that the target operator may determine the suitable location of the surgical device quickly and accurately.

In some embodiments, in order to facilitate the surgery of the patient, one or more markers may be set on the patient. For example, one or more markers may be placed on the body surface of the patient. The processing device 160A may obtain a fourth optical image of the one or more markers captured by a fourth visual sensor when the first optical image is captured. The fourth optical image may include information regarding, for example, the appearance of the one or more markers, the positions of the one or more markers relative to the patient, or the like, or any combination thereof. The processing device 160A may process the target image by adding a visual element representing the one or more markers on the target image based on the fourth optical image of the one or more markers. The visual element representing the one or more markers may include one or more icons in the target image, regions in shapes of the one or more markers, one or more real images of the one or more markers, etc.

In some embodiments, the visual element representing the one or more markers may be added to the target image in a similar manner as how the visual element representing the surgical device is added to the target image. In this way, the one or more markers placed on the patient may be displayed on the target image, so that the target operator may quickly determine relative positions of the one or more markers and organs and/or tissues in the target region, which may facilitate quickly and accurately determine a location of the surgery, thereby improving accuracy and efficiency of the surgery.

In some embodiments, one or more visual elements representing one or more other objects (e.g., the hands of the target operator) may also be added to the target image in a similar manner as how the visual element representing the surgical device is added to the target image.

In 408, the processing device 160A (e.g., the directing module 308) may direct a display device to display the rendered target image.

For example, the processing device 160A may transmit the rendered target image to the display device (e.g., the display device 140) via a network, and the display device may display the rendered target image to the target operator and/or other operators in the surgery room. More descriptions regarding the display device may be found elsewhere in the present disclosure. See, e.g., FIG. 2 and relevant descriptions thereof.

As described elsewhere in this disclosure, conventional approaches for visualizing an anatomical structure of a patient during a surgery often involve multiple medical scans performed on the patient before and during the surgery, or align the anatomical image of the patient to the body surface of the patient in the surgery based on markers on a surface of the patient.

According to some embodiments of the present disclosure, the anatomical image may be transformed based on a reference optical image and the first optical image such that a posture of the patient in the transformed anatomical image aligns with the posture of the patient in the first optical image, and the target image may be generated by combining the transformed anatomical image on the first optical image. In this way, the effect of the posture difference between the patient posture in the anatomical image and the patient posture in the surgery may be reduced or eliminated based on the first optical image and the reference optical image of the patient, thereby improving the quality of the target image which is the combination of the first optical image and the anatomical image. The target image may illustrate both the internal structure and the body surface of the patient, and can provide better guidance for the surgery than pure anatomical images or pure optical images.

In addition, the target image may be rendered according to the viewpoint of the target operator, and the rendered target image may reflect the real surgical scene seen by the target operator, therefore, the target operator may quickly perform an appropriate next surgical operation according to the rendered target image.

Compared with conventional approaches that involve multiple medical scans before and during the surgery, the methods of the present disclosure may reduce a count of the medical scans, thereby reducing adverse effects (e.g., radiation dosage) on the patient, and improving the efficiency of the generation of the target image. Moreover, the methods of the present disclosure do not require the patient to keep the same pose in the surgery as he/she was during the pre-operative medical scan. Compared with conventional approaches that align the anatomical image of the patient to the body surface of the patient in the surgery based on markers on the surface of the patient, the methods of the present disclosure based on optical image(s) may have an improved accuracy because the optical image(s), such as depth images and/or point cloud image(s), can provide more accurate information for reducing or eliminating the effect of the posture difference.

In some embodiments, the rendered target image may be displayed to the target operator by a display device instead of an augmented reality headset, which may be more convenient for the target operator to perform the surgery, and improve the user experience and the overall efficacy of the surgery guidance.

FIG. 5 is a flowchart illustrating an exemplary process 500 for transforming an anatomical image of a patient according to some embodiments of the present disclosure. In some embodiments, one or more operations of the process 500 may be performed to achieve at least part of operation 404 as described in connection with FIG. 4.

In 502, the processing device 160A (e.g., the generation module 304) may determine a first transformation relationship between a reference optical image and the anatomical image.

The anatomical image may be captured by performing a medical scan on the patient before or during the surgery, the reference optical image may be captured during the medical scan by a fifth visual sensor (e.g., a visual sensor 130). A field of view of the fifth visual sensor may cover the scanned region of the patient. In some embodiments, the reference optical image and the first optical image may include a same region of the patient. Since the reference optical image is captured during the medical scan, that is, the reference optical image and the anatomical image are captured simultaneously, a posture of the patient in the anatomical image and a posture of the patient in the reference optical image are substantially the same. Therefore, the processing device 160A may transform the anatomical image based on the reference optical image and the first optical image such that a posture of the patient in the transformed anatomical image aligns with the posture of the patient in the first optical image.

The first transformation relationship between a reference optical image and the anatomical image may indicate a corresponding relationship between points in the reference optical image and points in the anatomical image. For example, the first transformation relationship may be used to transform a coordinate of a first point in the reference optical image to a coordinate of a second point in the anatomical image, wherein the first point and the second point may correspond to the same physical point of the patient. In some embodiments, the first transformation relationship may be represented by a first transformation matrix.

In some embodiments, the processing device 160A may determine a transformation relationship between a third coordinate system corresponding to the reference optical image (e.g., a camera coordinate system of the fifth visual sensor) and a fourth coordinate system corresponding to the anatomical image (e.g., an imaging coordinate system of the medical imaging device). In some embodiments, the transformation relationship between the third coordinate system and the fourth coordinate system may be determined based on the spatial correlation between the fifth visual sensor and the medical imaging device. In some embodiments, the transformation relationship between the third coordinate system and the fourth coordinate system may be previously generated and stored in a storage device (e.g., a storage device of the surgery system 200, or an external source). The processing device 160A may retrieve the transformation relationship between the third coordinate system and the fourth coordinate system from the storage device. The processing device 160A may designate the transformation relationship between the third coordinate system and the fourth coordinate system as the first transformation relationship.

In 504, the processing device 160A (e.g., the generation module 304) may determine a second transformation relationship between the reference optical image and the first optical image.

The second transformation relationship between the reference optical image and the first optical image may indicate a corresponding relationship between points in the reference optical image and points in the first optical image. For example, the second transformation relationship may be used to transform a coordinate of a third point in the reference optical image to a coordinate of a fourth point in the first optical image, wherein the third point and the fourth point may correspond to the same physical point of the patient. In some embodiments, the second transformation relationship may be represented by a second transformation matrix.

In some embodiments, the processing device 160A may determine a reference representation relating to a posture of the patient in the reference optical image based on the reference optical image. The processing device 160A may also determine a target representation relating to the posture of the patient in the first optical image based on the first optical image.

A representation relating to a posture of the patient in an image may be denoted by characteristic information of the patient in the image. Exemplary characteristic information of the patient in the image may include posture information, shape information, position information, size information, direction information, or the like, or any combination thereof. In some embodiments, the representation of the patient may be represented as a 3D point cloud, a 3D mesh surface, one or more vectors, etc. The 3D point cloud may include a plurality of points, each of which may represent a spatial point on a body surface of the patient and be described using one or more feature values of the spatial point (e.g., feature values relating to the position (e.g., 3D coordinates) and/or the composition of the spatial point). The 3D mesh surface may include a collection of vertices, edges, and faces that defines a 3D shape of the patient. The vector(s) may include posture parameters and/or body parameters of the patient, such as a chest circumference, a head circumference, a height, a weight, a posture, a position, etc. In some embodiments, the vector(s) may include complicated features of the patient extracted by machine learning techniques. The vector(s) may include relatively small amounts of data compared with other forms of representations, which may improve the efficiency of the determination of the representation of the patient.

In some embodiments, the reference representation and the target representation may be determined in various manners. For illustration purposes, the determination of the reference representation is described as an example. For example, the processing device 160A may extract a 3D mesh surface from the reference optical image using a marching cube algorithm, and designate the 3D mesh surface as the reference representation. As another example, the processing device 160A may obtain the posture parameters and/or body parameters of the patient based on the reference optical image. The processing device 160A may generate one or more reference vectors according to the posture parameters and/or body parameters of the patient, and designate the one or more reference vectors as the reference representation. As still another example, the processing device 160A may obtain a reference 3D point cloud based on the reference optical image. A dimensionality reduction operation may be performed on the reference 3D point cloud of the patient to obtain the reference vector(s). For example, the processing device 160A may perform feature extraction on the reference 3D point cloud using an algorithm such as a local outlier factor algorithm (LOF), etc.

In some embodiments, the reference representation may be obtained using a representation determination model. The representation determination model may be a trained model (e.g., a machine learning model) configured to receive an image of a subject as an input, and output a representation relating to the posture of the subject in the image. Merely by way of example, the reference optical image of the patient may be inputted into the representation determination model, and the representation determination model may output the reference representation of the patient or information relating to the reference representation of the patient. In some embodiments, the representation determination model may include a deep learning model, such as a Deep Neural Network (DNN) model, a Convolutional Neural Network (CNN) model, a Recurrent Neural Network (RNN) model, a Feature Pyramid Network (FPN) model, etc. Exemplary CNN models may include a V-Net model, a U-Net model, a Link-Net model, or the like, or any combination thereof. In some embodiments, the representation determination model may be a portion of a trained model. For example, the representation determination model may be an encoder part of the trained model configured to extract characteristic information from an optical image to generate a representation of a subject in the optical image.

In some embodiments, the processing device 160A may obtain the representation determination model from one or more components of the surgery system 200 (e.g., the storage device 180) or an external source via a network. For example, the representation determination model may be previously trained by a computing device (e.g., the processing device 160B), and stored in the storage device 180. The processing device 160A may access the storage device 180 and retrieve the representation determination model. In some embodiments, the representation determination model may be generated according to a machine learning algorithm as described elsewhere in this disclosure (e.g., FIG. 4B and the relevant descriptions). More descriptions for the generation of the representation determination model may be found elsewhere in the present disclosure (e.g., FIG. 6 and the descriptions thereof).

After the reference representation and the target representation are determined, the processing device 160A may further determine the second transformation relationship based on the reference representation and the target representation of the patient. For example, the reference representation may include a first 3D point cloud and the target representation may include a second 3D point cloud. The second transformation relationship may be determined by registering the first 3D point cloud with the second 3D point cloud using a point cloud registration algorithm. Exemplary point cloud registration algorithms may include an iterative closest point (ICP) algorithm, a kernel correlation (KC) algorithm, a robust point matching (RPM) algorithm, an unscented particle filter (UPF) algorithm, an unscented Kalman filter (UKF) algorithm, etc. As another example, the reference representation may include one or more first vectors, and the target representation may include one or more second vectors. The processing device 160A may generate a first 3D model of the patient according to the first vector(s), and a second 3D model of the patient according to the second vector(s). The second transformation relationship may be determined by registering the first 3D model with the second 3D model.

In some embodiments, the processing device 160A may determine a plurality of feature point pairs. Each pair of the plurality of feature point pairs may include a first feature point in the reference optical image and a second feature point in the first optical image. Each of the plurality of feature point pairs may correspond to the same physical point of the patient. In some embodiments, the processing device 160A may determine the plurality of feature point pairs based on the target region of the patient and optionally surrounding regions of the target region. For example, the processing device 160A may determine select feature point pairs corresponding to points that locate on a boundary of the target region as a portion of the plurality of feature point pairs. The processing device 160A may determine the second transformation relationship based on the plurality of feature point pairs. For example, for each pair of the plurality of feature point pairs, the processing device 160A may determine a coordinate of a first feature point in the reference optical image and a coordinate of a second feature point in the first optical image. The processing device 160A may determine the second transformation relationship according to coordinates of feature points of the plurality of feature point pairs.

In 506, the processing device 160A (e.g., the generation module 304) may transform the anatomical image based on the first transformation relationship and the second transformation relationship such that the posture of the patient in the transformed anatomical image aligns with the posture of the patient in the first optical image.

In some embodiments, the processing device 160A may determine a third transformation relationship between the anatomical image and the first optical image based on the first transformation relationship and the second transformation relationship. Merely by way of example, if the first transformation relationship is represented as the first transformation matrix and the second transformation relationship is represented as the second transformation matrix, the processing device 160A may determine the third transformation matrix by multiplying the first transformation matrix with the second transformation matrix. The processing device 160A may further transform the anatomical image based on the third transformation relationship.

FIG. 6 is a flowchart illustrating an exemplary process 600 for generating a representation determination model according to some embodiments of the present disclosure. In some embodiments, the process 600 may be implemented in the surgery system 200 illustrated in FIG. 2. For example, the process 600 may be stored in a storage device of the surgery system 200 as a form of instructions, and invoked and/or executed by the processing device 160B (e.g., one or more modules as illustrated in FIG. 3B). The operations of the illustrated process presented below are intended to be illustrative. In some embodiments, the process 600 may be accomplished with one or more additional operations not described, and/or without one or more of the operations discussed. Additionally, the order in which the operations of the process 600 as illustrated in FIG. 6 and described below is not intended to be limiting.

In 602, the processing device 160B (e.g., the acquisition module 310) may obtain one or more training samples each of which includes a sample optical image of a sample subject.

As used herein, the sample subject may include a biological subject and/or a non-biological subject. For example, the sample subject may include a specific portion of a body, such as the head, the thorax, the abdomen, or the like, or a combination thereof. As another example, the sample subject may be a man-made composition of organic and/or inorganic matters that are with or without life. The sample optical image of the sample subject refers to an image illustrating an external surface of the sample subject (or a portion thereof). The sample optical image may include a 2D image, a 3D image, or the like. The sample optical image may be captured by a sample visual sensor.

In some embodiments, the processing device 160B may obtain a training sample (or a portion thereof) from one or more components of the surgery system 200 (e.g., the visual sensor(s) 130) or an external source (e.g., a database of a third-party) via a network.

In 604, the processing device 160B (e.g., the model generation module 312) may generate the representation determination model by training a preliminary model using the one or more training samples.

The preliminary model refers to a model to be trained. The preliminary model may be of any type of model (e.g., a machine learning model) as described elsewhere in this disclosure (e.g., FIG. 5 and the relevant descriptions). In some embodiments, the processing device 160B may obtain the preliminary model from one or more components of the surgery system 200 or an external source (e.g., a database of a third-party) via a network. The preliminary model may include a plurality of model parameters. Before training, the model parameters of the preliminary model may have their respective initial values. For example, the processing device 160B may initialize parameter values of the model parameters of the preliminary model.

In some embodiments, the preliminary model includes an encoder part and a decoder part, and an output of the encoder part may be input into the decoder part. For a training sample, the encoder part of the preliminary model may be configured to extract sample characteristic information (e.g., sample feature vectors) from the sample optical image of the training sample to generate a predicted representation of the sample subject in the sample optical image; and the decoder part of the preliminary model may be configured to generate a predict optical image based on the predicted representation of the sample subject in the sample optical image. Merely by way of example, the sample optical image of the training sample may be a sample 3D point cloud. The sample 3D point cloud may be input into the encoder part of the preliminary model, and the encoder part of the preliminary model may output one or more sample feature vectors. The sample feature vectors may be input into the decoder part of the preliminary model, and the decoder part of the preliminary model may output a predicted 3D point cloud of the sample subject.

In some embodiments, the model training process may include a plurality of iterations. Merely by way of example, in the current iteration, for each training sample, the sample optical image may be input into the encoder part of an intermediate model. The intermediate model may be the preliminary model in the first iteration of the plurality of iterations, or an updated model generated in a previous iteration. The encoder part of an intermediate model may output a predicted representation of the sample subject in the sample optical image. The predicted representation may be input into the decoder part of the intermediate model, and the decoder part of the intermediate model may output a predicted optical image. The processing device 160B may determine a value of a loss function based on the predicted optical image and the sample optical image of each training sample. The processing device 1606 may update at least one parameter of the intermediate model based on the value of the loss function.

The processing device 140B may then determine an assessment result of the updated intermediate model based on the value of the loss function. The assessment result may indicate whether the updated intermediate model is sufficiently trained. For example, the processing device 1606 may determine whether a termination condition is satisfied in the current iteration based on the value of the loss function. An exemplary termination condition may be that the value of the loss function in the current iteration is less than a threshold value, a difference between the values of the loss function obtained in a previous iteration and the current iteration (or among the values of the loss function within a certain number or count of successive iterations) is smaller than a certain threshold, or the like, or any combination thereof. Other exemplary termination conditions may include that a maximum number (or count) of iterations has been performed. In response to determining that the termination condition is not satisfied in the current iteration, the processing device 1606 may determine that the updated intermediate model is not sufficiently trained, and further update the updated intermediate model based on the value of the loss function. Merely by way of example, the processing device 1606 may update at least some of the parameter values of the updated intermediate model according to a backpropagation algorithm, e.g., a stochastic gradient descent backpropagation algorithm. The processing device 1606 may further perform a next iteration until the termination condition is satisfied. In response to determining that the termination condition is satisfied in the current iteration, the processing device 160B may determine that the updated intermediate model is sufficiently trained and terminate the training process. The encoder part of the updated intermediate model may be designated as the representation determination model.

It will be apparent to those skilled in the art that various changes and modifications can be made in the present disclosure without departing from the spirit and scope of the disclosure. In this manner, the present disclosure may be intended to include such modifications and variations if the modifications and variations of the present disclosure are within the scope of the appended claims and the equivalents thereof.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “module,” “unit,” “component,” “device,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate a certain variation (e.g., ±1%, ±5%, ±10%, or ±20%) of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable. In some embodiments, a classification condition used in classification or determination is provided for illustration purposes and modified according to different situations. For example, a classification condition that “a value is greater than the threshold value” may further include or exclude a condition that “the probability value is equal to the threshold value.”

What is claimed is:

1. A system, comprising:
at least one storage device including a set of instructions; and
at least one processor configured to communicate with the at least one storage device, wherein when executing the set of instructions, the at least one processor is configured to direct the system to perform operations including:
obtaining an anatomical image and a first optical image of a patient, the anatomical image being captured by performing a medical scan on the patient before a surgery of the patient, and the first optical image being captured during the surgery;
generating a target image by combining the anatomical image and the first optical image;
rendering the target image based on a viewpoint of a target operator of the surgery; and
directing a display device to display the rendered target image.

2. The system of claim 1, wherein the generating a target image by combining the anatomical image and the first optical image includes:
obtaining a reference optical image of the patient captured during the medical scan;
transforming the anatomical image based on the reference optical image and the first optical image such that a posture of the patient in the transformed anatomical image aligns with the posture of the patient in the first optical image; and
generating the target image by combining the transformed anatomical image and the first optical image.

3. The system of claim 2, wherein the transforming the anatomical image based on the reference optical image and the first optical image includes:
determining a first transformation relationship between the reference optical image and the anatomical image;
determining a second transformation relationship between the reference optical image and the first optical image; and
transforming the anatomical image based on the first transformation relationship and the second transformation relationship such that the posture of the patient in the transformed anatomical image aligns with the posture of the patient in the first optical image.

4. The system of claim 3, wherein the determining a second transformation relationship between the reference optical image and the first optical image includes:
determining, based on the reference optical image, a reference representation relating to the posture of the patient in the reference optical image;
determining, based on the first optical image, a target representation relating to the posture of the patient in the first optical image; and
determining, based on the reference representation and the target representation of the patient, the second transformation relationship.

5. The system of claim 4, wherein the reference representation and the target representation of the patient are obtained using a representation determination model.

6. The system of claim 2, wherein the generating the target image by combining the transformed anatomical image and the first optical image includes:
determining, based on the viewpoint of the target operator, a field of view (FOV) of the target operator;
determining a target portion of the transformed anatomical image corresponding to at least a portion of the patient within the FOV of the target operation; and
generating the target image by overlaying the target portion of the transformed anatomical image on the first optical image.

7. The system of claim 1, wherein determining the viewpoint of the target operator includes:
determining facial information of the target operator based on a second optical image of the target operator; and
determining, based on the facial information, the viewpoint of the target operator.

8. The system of claim 1, wherein the rendering the target image based on a viewpoint of a target operator of the surgery includes:
obtaining a third optical image of a surgical device used in the surgery captured when the first optical image is captured;
processing the target image by adding a visual element representing the surgical device on the target image based on the third optical image of the surgical device; and
rendering the processed target image based on the viewpoint of the target operator.

9. The system of claim 1, wherein the rendering the target image based on a viewpoint of a target operator of the surgery includes:
obtaining a fourth optical image of one or more markers placed on the patient captured when the first optical image is captured; and
processing the target image by adding a visual element representing the one or more markers on the target image based on the fourth optical image of the one or more markers; and
rendering the processed target image based on the viewpoint of the target operator.

10. The system of claim 1, wherein the viewpoint of the target operator is set by the target operator.

11. A method, the method being implemented on a computing device having at least one storage device and at least one processor, the method comprising:
obtaining an anatomical image and a first optical image of a patient, the anatomical image being captured by performing a medical scan on the patient before a surgery of the patient, and the first optical image being captured during the surgery;
generating a target image by combining the anatomical image and the first optical image;
rendering the target image based on a viewpoint of a target operator of the surgery; and
directing a display device to display the rendered target image.

12. The method of claim 11, wherein the generating a target image by combining the anatomical image and the first optical image includes:
obtaining a reference optical image of the patient captured during the medical scan;
transforming the anatomical image based on the reference optical image and the first optical image such that a posture of the patient in the transformed anatomical image aligns with the posture of the patient in the first optical image; and
generating the target image by combining the transformed anatomical image and the first optical image.

13. The method of claim 12, wherein the transforming the anatomical image based on the reference optical image and the first optical image includes:
determining a first transformation relationship between the reference optical image and the anatomical image;
determining a second transformation relationship between the reference optical image and the first optical image; and
transforming the anatomical image based on the first transformation relationship and the second transformation relationship such that the posture of the patient in the transformed anatomical image aligns with the posture of the patient in the first optical image.

14. The method of claim 13, wherein the determining a second transformation relationship between the reference optical image and the first optical image includes:
determining, based on the reference optical image, a reference representation relating to the posture of the patient in the reference optical image;
determining, based on the first optical image, a target representation relating to the posture of the patient in the first optical image; and
determining, based on the reference representation and the target representation of the patient, the second transformation relationship.

15. The method of claim 14, wherein the reference representation and the target representation of the patient are obtained using a representation determination model.

16. The method of claim 12, wherein the generating the target image by combining the transformed anatomical image and the first optical image includes:
determining, based on the viewpoint of the target operator, a field of view (FOV) of the target operator;
determining a target portion of the transformed anatomical image corresponding to at least a portion of the patient within the FOV of the target operation; and
generating the target image by overlaying the target portion of the transformed anatomical image on the first optical image.

17. The method of claim 11, wherein determining the viewpoint of the target operator includes:
determining facial information of the target operator based on a second optical image of the target operator; and
determining, based on the facial information, the viewpoint of the target operator.

18. The method of claim 11, wherein the rendering the target image based on a viewpoint of a target operator of the surgery includes:
obtaining a third optical image of a surgical device used in the surgery captured when the first optical image is captured;
processing the target image by adding a visual element representing the surgical device on the target image based on the third optical image of the surgical device; and
rendering the processed target image based on the viewpoint of the target operator.

19. The method of claim 11, wherein the rendering the target image based on a viewpoint of a target operator of the surgery includes:
obtaining a fourth optical image of one or more markers placed on the patient captured when the first optical image is captured; and
processing the target image by adding a visual element representing the one or more markers on the target image based on the fourth optical image of the one or more markers; and
rendering the processed target image based on the viewpoint of the target operator.

20. A non-transitory computer readable medium, comprising at least one set of instructions, wherein when executed by one or more processors of a computing device, the at least one set of instructions causes the computing device to perform a method, the method comprising:
obtaining an anatomical image and a first optical image of a patient, the anatomical image being captured by performing a medical scan on the patient before a surgery of the patient, and the first optical image being captured during the surgery;
generating a target image by combining the anatomical image and the first optical image;
rendering the target image based on a viewpoint of a target operator of the surgery; and
directing a display device to display the rendered target image.
